(19) United States
(12) Patent Application Publication
(10) Pub. No.: US 2005/0148920 A1
(43) Pub. Date: Jul. 7, 2005

(54) Polyurethane foam cavity wound dressings

(76) Inventor: Deborah Addison, Via Lancaster (GB)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/498,921

(22) PCT Filed: Dec. 13, 2002
(86) PCT No.: PCT/GB02/05656

(30) Foreign Application Priority Data
Dec. 14, 2001 (GB) 0129974.2

(51) Int. Cl. A61F 15/00; A61F 13/00

(52) U.S. Cl. 602/46

(57) ABSTRACT

A wound dressing comprising an elongate spiral roll of a polyurethane foam. Also provided is a method of preparing such dressings comprising the steps of: providing a foaming mixture comprising an isocyanate-capped prepolymer and a foaming agent; casting the foaming mixture into a sheet; and rolling up the sheet into a spiral roll before curing of the polyurethane is complete.

The present invention relates to cavity wound dressings made from polyurethane foams, and to methods for the manufacture of such wound dressings.

There is a need in clinical practice for absorbent medicinal articles that can be inserted into deep wounds to absorb wound exudate and provide cavity filling. Such dressings should be highly absorbent, conformable to the shape of the wound, and inexpensive.

It is known to provide textile or foam-based absorbent structures for dressing deep wounds. Those structures are insufficiently conformable to fit every deep wound, unless they are cut into shape. Furthermore, such absorbent structures can be relatively difficult to remove from a deep wound after use without causing further trauma.

U.S. Pat. No. 4,837,285 describes the use for soft tissue cavity filling of resorbable collagen sponge beads having diameters of from 0.1 to 4.0 mm. The beads have pore sizes of from 50 to 350 micrometers. Such beads can be difficult to handle, and are not suitable for use as a removable, absorbent dressing for deep wounds.

DE-A-4037931 describes a deep medicinal article structure consisting of a cavity filling plug of resorbable collagen foam containing an array of hollow resorbable filaments. The filaments are bundled together at one end, and connected to a drain for wound fluid, whereby the wound fluid is continuously drained from the wound cavity through the hollow filaments. Such a structure is relatively expensive to construct, and insufficiently conformable to a wide range of wound cavity shapes.

EP-A-0171268 describes absorbent, non-adherent wound dressings for use in the treatment of deep wounds, wherein the dressing comprises a porous bag containing individual pieces of a conformable, resilient, absorbent hydrophilic foam. The porous bag may be provided with a string to assist removal of the bag from the wound after use.

U.S. Pat. No. 5,885,237 describes a wound dressing in the form of a thin wafer or disc. The wafer is provided with a spiral line of weakness to facilitate the unraveling of the disc or wafer to provide a strand or rope of dressing material for wound filling or covering.

It is known to use hydrophilic polyurethane foams as absorbent wound dressing materials. U.S. Pat. No. 4,339,550 and EP-A-0335669 describe suitable hydrophilic foam compositions formed by the reaction of isocyanate-capped polyether prepolymers with water and other agents. The water acts as a foaming and cross-linking agent.

WO99/02587 describes a variety of isocyanates and isocyanate capped prepolymers suitable for the manufacture of polyurethane wound dressing foams.

EP-A-0541391 describes a method of making conformable, high-density polyurethane foams, and the use of such foams as wound contacting layers in wound dressings. The foams are made by mixing 1 part by weight of an isocyanate-capped prepolymer having from 0.5 to 1.2 meq NCO groups/g with from 0.4 to 1.0 parts by weight of water in the presence of from 0.05 to 0.4 parts by weight of C₁ to C₃ monohydric alcohol, and then drying the product. The resulting foams are highly absorbent, stretchable and conformable. They present an excellent wound contacting surface, and are currently used as such in island-type wound dressings under the Registered Trade Mark TIELLE.

Hitherto, polyurethane foam wound dressings have been produced in thin layers and strips. It can be difficult to produce uniformly foamed strips of thickness greater than a few millimeters. The thin polyurethane foam layers can exhibit low tensile strength and excessive swelling when wet, which makes them less suitable for packing deep wounds and cavities.

It is an objective of the present invention to provide improved absorbent dressings for a deep wounds and soft tissue cavity filling.

The present invention provides a wound dressing comprising an elongate spiral roll of a polyurethane foam.

The term “spiral roll” refers to a structure formed by rolling up a flat layer of foam into a “swiss roll” having a plurality of layers arranged in spiral fashion around an axis. The plurality of layers are bonded together to form an integral structure. The bonding may be by means of a layer of adhesive, such as a hydrocolloid adhesive, on the layers. Preferably, the bonding is by means of covalent urethane or urea linkages between layers, for example formed by rolling up a sheet of polyurethane foam into the spiral before curing of the polyurethane is complete. The spiral roll may thereby comprise alternating layers of polyurethane foam and polyurethane gel.

Preferably, the spiral roll comprises from 2 to about 20 layers of the polyurethane foam measured across the diameter of the roll, more preferably from about 4 to about 10 layers. Typical layer thicknesses are from about 0.5 mm to about 4 mm, preferably from about 0.8 mm to about 2 mm, and most preferably about 1 mm. Accordingly, the diameter of the spiral roll is preferably from about 2 to about 20 mm, more preferably from about 4 to about 10 mm.

The spiral roll is elongate. That is to say, the length of the roll is greater than its maximum diameter. Typically, the length of the spiral roll is from about 20 mm to about 1000 mm, preferably from about 50 mm to about 300 mm. The spiral roll can conveniently be cut into suitable lengths for a given wound before use.

In certain embodiments the wound dressing consists essentially of the polyurethane foam material. It will be appreciated that in such embodiments the foam may not be completely physically homogeneous, so there may be a layer of unfoamed material or compressed foam on the surfaces of the foam that is rolled up with the foamed layer. In other embodiments the spiral roll comprises one or more further layers rolled up with the polyurethane layer, for example layers of medicaments or superabsorbent layers. In some embodiments the spiral roll may comprise a pull thread rolled up with the polyurethane layers and extending out of one end of the roll to assist removal of the dressing after use.

The polyurethane foam material is highly flexible, absorbent and conformable to different wound shapes. Preferably, the spiral roll has a density of at least 0.28 g/cm³, and more preferably at least 0.30 g/cm³. Particularly preferred rolls have a density in the range 0.3 to 1.0 g/cm³, e.g. about 0.5 g/cm³. Preferably, the spiral roll material (and hence the dressing) has a wet tensile strength (measured immediately after saturation with water) of from about 100 g to about 2000 g, preferably greater than about 200 g and most preferably greater than about 500 g. Typically this is achieved by a roll of diameter about 5 mm. This makes the spiral roll material especially easy to remove from the wound in one piece after use.

Preferably, the spiral roll has an elongation at break of at least about 50%.

Depending on the proportions of other additives, the wound dressings of the invention preferably have an absorbency of at least about 3 g saline/g, preferably at least about 5 g/g, and more preferably at least about 8 g/g. The dressings are thus highly absorbent, yet conformable.

The dressings of the invention also have the property of swelling and expanding when water is absorbed, but to a lesser extent than polyurethane foam layers that have not been formed into a spiral roll. This is particularly advantageous in a deep wound, because the swelling of the foam causes it to move inwards towards the wound bed thus filling the wound cavity, but without swelling excessively. This encourages the wound to heal from the base upwards and outwards, and it discourages epithelialization over the wound surface before the bed has been filled with granulation tissue.

The degree of swelling of the spiral rolls the present invention on complete saturation with an aqueous medium is typically about 50% to 600% (expressed in terms of increase in volume), and preferably about 100% to about 400%. Despite this high degree of swelling, however the foams used in the invention retain their integrity even after absorption of large quantities of water.

Preferably, the foams have a morphology which is particularly appropriate for low adherence wound dressings. The foams are preferably open-celled, the cells being very regular in size and shape, with very smooth edges to the pores in the walls of the cells. Typically, the cells of the foams of the invention have an average diameter in the range 0.1 to 0.6 mm.

The spiral roll in the wound dressings of the invention may also include topical medicaments and antiseptics, such as silver sulfadiazine, povidone iodine, chlorhexidine acetate and chlorhexidine gluconate, as well as other therapeutically useful additives such as polypeptide growth factors and enzymes.

The spiral roll wound dressings of the invention preferably comprise less than 10% by weight of water, more preferably less than 2% by weight of water, and hence they are easy to sterilized by gamma irradiation or other methods. The dressings are preferably sterile and packaged in a microorganism-impermeable container.

The present invention further provides a method of manufacture of a wound dressing comprising the steps of:
providing a foaming mixture comprising an isocyanate capped prepolymer and a foaming agent;
casting the foaming mixture into a sheet; and
rolling up the sheet into a spiral roll before curing of the polyurethane is complete.

Suitable isocyanate capped prepolymers and methods of manufacture thereof are described in EP-A-0335669 and WO9902587, the entire contents of which are incorporated herein by reference.

Preferably, the isocyanate-capped prepolymer comprises from 0.5 to 1.2 meq NCO groups/g. Preferably, the isocyanate-capped prepolymer is an isocyanate-capped polyether prepolymer. More preferably, the prepolymer is an isocyanate-capped oxy ethylene oxy/propylene copolymer. Suitable prepolymers are available under the Registered Trade Mark HYPOL from Dow Chemical Company, 2 Heathrow Boulevard, 284 Bath Road, West Drayton, U.K.

Preferably, the foaming agent comprises water. The water reacts with the isocyanate prepolymer to cross-link the isocyanate groups in urea linkages and release CO₂, which causes the polyurethane reaction mixture to form a foam.

Preferably, the foaming mixture further comprises a polyurethane or polyurea chain terminating compound, preferably selected from monohydric alcohols and amines.

Certain preferred methods of the invention comprehend the use of any of methanol, ethanol or propanol, and the use of methanol is particularly preferred. All three alcohols reduce the rate of reaction between the isocyanate-capped prepolymer and water, but the effect of methanol is more marked. A reduction of the reaction rate is desirable in order to facilitate mixing of the various components and spreading of the reaction mixture into a layer of suitable thickness for curing. In addition, the monohydric alcohol serves to end cap some of the NCO end groups, preventing reaction with water to form the urea linkage. This also gives a more flexible, conformable foam.

More preferably, one part by weight of the isocyanate-capped prepolymer is mixed with water in the presence of from 0.05 to 0.25 parts by weight of methanol or from 0.1 to 0.3 parts by weight of ethanol.

The amounts of chain extending and terminating compounds influence the physical properties of the foam. In particular, amine/diamine chain terminating/extending compounds react with the prepolymer very much faster than water and methanol. For example, the tackiness of the foam increases with increasing monohydric alcohol or monoamine content in the mixture. Preferably, the chain extending and terminating compounds are present in the mixture in an amount of 0.05 to 0.4 parts for alcohols and 0.01 to 0.05 parts for the amines, based on one part by weight of the isocyanate prepolymer.

In certain embodiments the foaming mixture further comprises a catalyst for the polymerisation, preferably a diamine such as diazobicyclo octane or dimethylaminoethyl ether. Preferably, the catalysts are present in the mixture in an amount of 0.005 to 0.02 parts by weight, based on one part by weight of the isocyanate prepolymer.

Preferably, the basis weight of the polyurethane/polyurea layer is from 0.2 to 1.5 kg/m², more preferably 0.5 to 1.0 kg/m². Preferably, the thickness of the layer is from 0.4 to 5 mm, more preferably from 0.5 to 2 mm. Preferably, the density of the final layer is at least 0.28 g/cm³, preferably at least 0.32 g/cm³.

The foaming mixture may comprise other conventional wound therapeutic materials. Suitable therapeutic materials include: antiseptics such as molecular silver, silver sulfadiazine or chlorhexidine; pain relieving agents such as lignocaine; anti-scarring agents such as mannose-6-phosphate, and agents for promoting wound healing such as growth factors.

Preferably, the foaming mixture comprises one part by weight of the isocyanate-capped prepolymer to 0.4 to 1.0 parts by weight of water. Preferably, the mixture also comprises 0.05 to 0.4 parts by weight of a C₁ to C₃ monohydric alcohol. Preferably, the mixture also comprises 0.01 to 0.03 parts by weight of one or more diamines.

Preferably, the process according to the present invention is carried out at ambient temperatures, i.e. 15 to 25° C. Preferably, for monohydric alcohol systems, the layers are allowed to foam and set for about 1 to about 100 minutes, preferably about 2 to about 8 minutes, before rolling up to form the spiral roll. Preferably, for systems containing diamines, the layers are allowed to foam and set for about 0.5 to about 10 minutes, more preferably for about 1 to about 3 minutes, before rolling up to form the roll.

Preferably, the water content of the product spiral roll is less than 1% by weight.

It will be appreciated that other components may be added to the reaction mixture in the method of the invention, in order to give desired properties to the product. In particular, it is preferable to include a small proportion (e.g. up to 30% by weight of the wet composition) of a rubber, which may be either natural or synthetic. This has the effect of increasing the cure time for the polyurethane, and increases extensibility, strength and tack. Most importantly, it substantially reduces shrinkage of the gel on drying, and it also improves bubble formation, producing more regular, smaller bubbles.

Preferably, the rubber is added in the form of a latex, ie. a suspension or emulsion of the rubber in an aqueous medium. The latex will generally comprise 40 to 70% solids by weight, e.g. 50 to 60% by weight. Since the foam is to be used as a wound contact layer, the rubber must be pharmaceutically acceptable.

Acrylic-based rubbers are particularly preferred. These are commercially available in the form of latexes, such as PRIMAL B-15J and RHOPLEX N-560 (Registered Trade Marks), manufactured by the Rohm & Haas company.

In addition to the methanol or ethanol, other alcohols, and particularly polyols, may be included in the foaming mixture to produce a softer, more conformable foam. For example, a polyol sold by Bayer AG under the Registered Trade Mark LEVAGEL may be used. However, traces of such alcohols are likely to remain in the free form after the foaming reaction, and these traces may be difficult to remove from the foam merely by heating. The use of higher boiling alcohols is therefore preferably avoided, because of the likelihood that such alcohols will be leached from the foam during use of the dressing. Preferably, the spiral rolls contain less than 1% by weight of water soluble alcohols, and more preferably less than 0.1% by weight It is particularly preferred that the spiral roll dressings of the invention are essentially free of water soluble alcohols (e.g. less that 0.01% by weight).

Specific embodiments of the present invention will now be described further, by way of example, with reference to the accompanying drawings, in which:

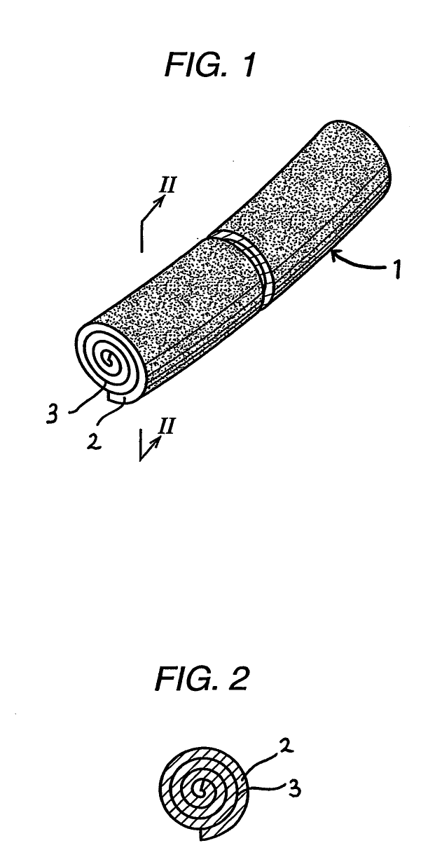

FIG. 1 shows a perspective view of a wound dressing according to the invention; and

FIG. 2 shows a transverse cross-section through the wound dressing of FIG. 1.

### EXAMPLE 1

Methanol (6 g) was added to HYPOL (Registered Trade Mark) Hydrogel prepolymer (50 g; NCO content 0.5-1.2 meq/g) in a disposable cup and mixed thoroughly for a few seconds. Water (32 g) and Primal B15-J Latex (12 g) were then added to the HYPOL mixture and stirred vigorously. The foaming mixture was poured onto release paper and spread using a stainless steel hand spreader set at a gap of 2.2 mm. The foam was left to cure partially for 90 seconds and then was rolled up by hand to form the spiral roll. The spiral roll was then placed in an oven at 80° C.-100° C. for 30 min to drive off the water and methanol. The finished roll could then be packaged and sterilized.

Referring to the drawings, the wound dressing comprises a single sheet 2 of the polyurethane foam about 1 mm thick rolled up into a spiral roll 1 of diameter about 6 mm. The slight physical inhomogeneity between the surface and interior of the foam layer means that there is a gel-like spiral interface 3 between successive layers in the spiral.

The spiral roll 1 is found to have a wet tensile strength greater about 600 g.

The above embodiment has been described by way of example only. Many other embodiments falling within the scope of the accompanying claims will be apparent to the skilled reader.

1. A wound dressing comprising an elongate spiral roll of a polyurethane foam.

2. A wound dressing according to claim 1, wherein the spiral roll comprises from 2 to about 20 layers of the polyurethane foam.

3. A wound dressing according to claim 1, wherein the diameter of the spiral roll is from about 2 to about 20 mm.

4. A wound dressing according to claim 1, wherein the length of the spiral roll is from about 20 mm to about 1000 mm.

5. A wound dressing according to claim 1, wherein the wound dressing consists essentially of the polyurethane foam material.

6. A wound dressing according to claim 1, wherein the polyurethane foam material has a density of at least 0.28 g/cm³, and an elongation at break of at least 50%.

7. A wound dressing according to any preceding claim, wherein the dressing is sterile and packaged in a microorganism-impermeable container.

8. A method of manufacture of a wound dressing comprising the steps of:
providing a foaming mixture comprising an isocyanate-capped prepolymer and a foaming agent;
casting the foaming mixture into a sheet; and
rolling up the sheet into a spiral roll before curing of the polyurethane is complete.

9. A method of manufacture according to claim 8, wherein the isocyanate-capped prepolymer comprises from 0.5 to 1.2 meq NCO groups/g.

10. A method of manufacture according to claim 8, wherein the foaming agent comprises water.

11. A method of manufacture according to claim 8, wherein the foaming mixture further comprises a polyurethane or polyurea chain terminating compound, selected from monohydric alcohols and amines.

12. A wound dressing obtainable by a method according to any one of claims 8 to 11.
